(19) United States
(12) Patent Application Publication
(10) Pub. No.: US 2026/0219199 A1
(43) Pub. Date: Jul. 30, 2026

(54) ROAD SURFACE CONDITION DETECTION APPARATUS AND METHOD FOR OPERATING THEREOF

(71) Applicant: Luxar Co., Daejeon (KR)

(72) Inventors: Sang Soo LEE, Cohoes, NY (US); Khalid AHMAD, Durham, NC (US); George BUABBUD, Lovettsville, VA (US)

(21) Appl. No.: 19/433,525

(22) Filed: Dec. 26, 2025

Related U.S. Application Data
(60) Provisional application No. 63/749,154, filed on Jan. 24, 2025.

(51) Int. Cl. G01N 21/88; G01N 21/3559; G01N 21/3563; G01N 21/3577; G01N 21/359; G01N 21/84; G01N 33/18

(52) U.S. Cl. CPC G01N 21/8806; G01N 21/3559; G01N 21/3563; G01N 21/3577; G01N 21/359; G01N 21/8422; G01N 33/1873; G01N 2021/8427; G01N 2021/8845

(57) ABSTRACT

A road surface condition detection apparatus is provided. The road surface condition detection apparatus includes a light source configured to irradiate light to a road surface, a detector configured to receive reflected light reflected by the road surface from the light irradiated by the light source, and a processor configured to control the light source and the detector and to detect whether the road is frozen based on the reflected light.

### CROSS-REFERENCE TO RELATED APPLICATIONS

The present application claims the benefit of and priority to U.S. Provisional Patent Application No. 63/749,154 filed Jan. 24, 2025, which is hereby incorporated by reference herein in its entirety.

### BACKGROUND

The present disclosure relates to a road surface condition detection apparatus, and more particularly, to an apparatus for detecting a state of a road surface by separating intrinsic absorption characteristics of a medium using a spectral-loss inversion scheme, and a method for operating the same.

Black ice is a transparent thin ice layer that is difficult to visually identify and is a major cause of loss of vehicle control and accidents by rapidly reducing friction between a tire and a road surface. To solve this problem, many techniques distinguish between water and ice by combining specific wavelengths based on the fact that water and ice have different absorption coefficients in a visible-to-short-wave infrared (VIS-SWIR) region. However, because reflectance of an actual road surface varies significantly with position and time due to factors such as material, roughness, and contamination, conventional spectroscopy-based approaches have a limitation in that it is difficult to maintain a consistent decision criterion.

### SUMMARY

A technical problem to be solved by the present invention is to remove variations in reflectance and diffuse-reflection characteristics of a road surface so as to purely separate only an intrinsic absorption component of a medium. An object of the present invention is to determine black ice based solely on the intrinsic absorption component of the medium.

Another technical problem to be solved by the present invention is to independently separate a reflection component from a surface of a medium based on Brewster reflection. The present invention aims to improve reliability of black ice detection based on the reflection component regardless of a type of the road surface.

According to one embodiment of the present disclosure, a road surface condition detection apparatus includes a light source configured to irradiate light to a road surface, a detector configured to receive reflected light reflected by the road surface from the light irradiated by the light source, and a processor configured to control the light source and the detector and to detect whether the road is frozen based on the reflected light. The light source includes a first light source configured to output light in a first wavelength band selected to exhibit inversion of absorption characteristics between water and ice, and a second light source configured to output light in a second wavelength band different from the first wavelength band. The processor is configured to normalize first reflected light corresponding to the first wavelength band and second reflected light corresponding to the second wavelength band with respect to a dry road surface, calculate an intensity ratio between the normalized first reflected light and the normalized second reflected light, and determine whether the road surface is frozen by comparing the calculated intensity ratio with a reference value.

In one embodiment, the light source irradiates short-wavelength infrared (SWIR) light, and the short-wavelength infrared light is parallel light having a predetermined diameter.

In one embodiment, the first wavelength band includes 1550 nm, and the second wavelength band includes 940 nm or 1380 nm.

In one embodiment, the reflected light includes first reflected light corresponding to light in the first wavelength band and second reflected light corresponding to light in the second wavelength band.

In one embodiment, the processor controls output intensities of the first light source and the second light source to be maintained constant over time.

In one embodiment, the processor controls output wavelengths of the first light source and the second light source to be maintained constant over time.

In one embodiment, the road surface condition detection apparatus further includes a scanner configured to move the light source and the detector, and the processor divides the road surface into a plurality of unit cells and controls the scanner to two-dimensionally scan irradiation points of the light source and the detector for the plurality of unit cells while determining whether each unit cell is frozen.

In one embodiment, the processor sets a size or an area of each unit cell to correspond to a diameter of the parallel light irradiated from the light source or an irradiation area by the parallel light.

In one embodiment, the processor controls the light source and the detector to sequentially scan the unit cells, and controls the detector to receive reflected light for at least a period corresponding to an integration time or an exposure time for each unit cell before moving to a next unit cell.

In one embodiment, the light source operates in a pulse mode or a continuous-wave mode, and the second light source outputs light in the second wavelength band after the first light source outputs light in the first wavelength band.

In one embodiment, the detector comprises an image sensor, and the image sensor separates light in the first wavelength band and light in the second wavelength band based on a wavelength-selective filter.

In one embodiment, the image sensor comprises a zoom lens or a telephoto lens.

In one embodiment, the processor controls the image sensor to selectively operate in one of a gated mode and a non-gated mode, wherein the gated mode controls an exposure time of the image sensor in correspondence with an emission timing of the light source.

In one embodiment, the road surface condition detection apparatus further includes a Brewster-reflection-based surface determination channel configured to determine whether the road surface is frozen using surface reflection characteristics of a medium formed on the road surface.

In one embodiment, the Brewster-reflection-based surface determination channel is configured such that the light source irradiates linearly polarized light toward the road surface, and the light source includes a polarizer disposed on an output optical path.

In one embodiment, the linearly polarized light is P-polarized light having an electric-field component parallel to an incident plane and is incident at an incident angle corresponding to a Brewster angle of the medium formed on the road surface.

In one embodiment, to form the incident angle corresponding to the Brewster angle, at least one of the light source or the detector includes at least one of an angle adjuster configured to adjust an irradiation angle or a height adjuster configured to adjust a relative installation height with respect to the road surface.

In one embodiment, the detector detects a P-polarized reflection component reflected from the road surface under a Brewster-reflection condition, and the processor determines whether the medium formed on the road surface is water or ice based on whether an intensity of the P-polarized reflection component vanishes or decreases.

In one embodiment, the processor combines a determination result from the Brewster-reflection-based surface determination channel with a freezing determination result based on absorption inversion and normalization to determine whether the road surface is frozen in an auxiliary or cross-validation manner.

In one embodiment, the processor determines whether each of a plurality of cells is frozen and maps a distribution of a surface layer film on the road surface based on determination results for the plurality of cells.

### BRIEF DESCRIPTION OF THE FIGURES

The above and other objects and features of the present disclosure will become apparent by describing in detail embodiments thereof with reference to the accompanying drawings.

FIG. 1 illustrates light transmitted through a water film or an ice film formed on a road surface.

FIG. 2A illustrates light reflected from a road surface.

FIG. 2B illustrates light reflected from a surface of a thin film formed on the road surface.

FIG. 2C illustrates light reflected from both the road surface and the thin film surface.

FIG. 3 is an example illustrating a difference in reflectance caused by road repair.

FIG. 4 illustrates a road surface condition detection apparatus according to one embodiment of the present disclosure.

FIG. 5 illustrates a road surface condition detection apparatus 100b according to another embodiment of the present disclosure.

FIG. 6 illustrates a road surface condition detection apparatus 100c according to another embodiment of the present disclosure.

FIG. 7A illustrates an example of dividing a road surface according to one embodiment of the present disclosure.

FIG. 7B illustrates an example of irradiating light in a first wavelength band onto the road surface of FIG. 7A.

FIG. 7C illustrates an example of irradiating light in a second wavelength band onto the road surface of FIG. 7A.

FIG. 8 is a graph illustrating changes in an imaginary part of a complex refractive index of water and ice according to wavelength.

FIG. 9 is a graph illustrating results measured for a dry road surface by the road surface condition detection apparatus according to one embodiment of the present disclosure.

FIG. 10 is a graph illustrating normalized values of measurement results for the dry road surface of FIG. 9.

FIG. 11A is a measurement image of first reflected light and second reflected light for the normalized dry road surface of FIG. 10.

FIG. 11B is a measurement image of first reflected light and second reflected light for a normalized water film of FIG. 10.

FIG. 11C is a measurement image of first reflected light and second reflected light for a normalized ice film of FIG. 10.

FIG. 12 is a graph illustrating an example of determining a road surface condition based on an intensity ratio of reflected light according to one embodiment of the present disclosure.

FIG. 13 is a schematic diagram illustrating an application example of a road surface condition detection apparatus according to one embodiment of the present disclosure.

FIG. 14 is a flowchart illustrating a method for operating a road surface condition detection apparatus according to one embodiment of the present disclosure.

### DETAILED DESCRIPTION

Hereinafter, embodiments of the present disclosure will be described clearly and in detail so that those skilled in the art to which the present disclosure pertains can easily carry out the disclosure.

Components described with reference to the terms unit, module, block, or suffixes such as -or and -er, as well as functional blocks shown in the drawings, may be implemented in the form of software, hardware, or a combination thereof. For example, the software may include machine code, firmware, embedded code, or application software. The hardware may include electrical circuits, electronic circuits, processors, computers, integrated circuits, integrated circuit cores, pressure sensors, inertial sensors, microelectromechanical systems (MEMS), passive elements, or combinations thereof. Hereinafter, in order to clearly describe the technical idea of the present invention, detailed descriptions of duplicated components will be omitted.

In the present document, each of the phrases “A or B”, “at least one of A and B”, “at least one of A or B”, “A, B, or C”, “at least one of A, B, and C”, and “at least one of A, B, or C” may include any one of the items listed together in the relevant phrase, or any possible combination of all of them.

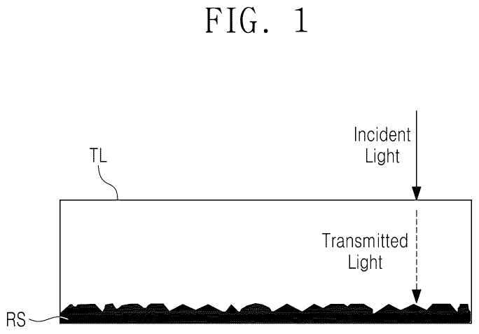

FIG. 1 illustrates light transmitted through a water film or an ice film formed on a road surface.

Referring to FIG. 1, a thin film TL may be formed on the road surface. For example, the thin film TL may include a water film or an ice film. The water film and the ice film may have optically different absorption coefficients. For example, the water film may have an absorption coefficient k (w), and the ice film may have an absorption coefficient k (i). Based on the different absorption coefficients, a difference in intensity of transmitted light at a specific wavelength may occur between the water film and the ice film.

A technique for detecting a road surface condition may detect a state of a road surface based on spectroscopic differences between a water film and an ice film. A road surface on which a water film or an ice film is formed may exhibit various reflection characteristics. A road surface on which a water film or an ice film is formed may form a complex reflection pattern based on a combination of intrinsic absorption characteristics of a medium, such as water or ice.

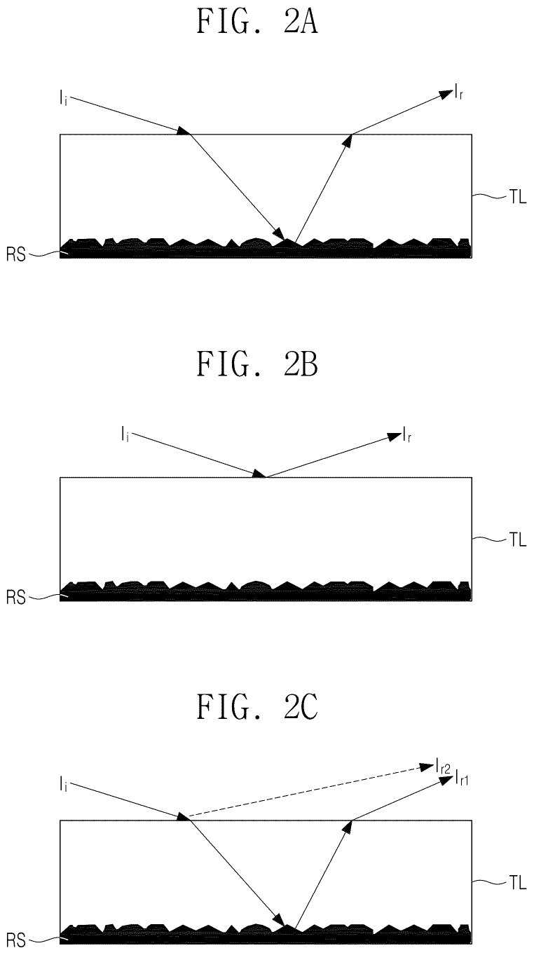

FIG. 2A illustrates light reflected from a road surface. FIG. 2B illustrates light reflected from a surface of a thin film formed on the road surface. FIG. 2C illustrates light reflected from both the road surface and the thin film surface.

Referring to FIG. 2A, reflection from the road surface may be classified as diffuse reflection. Incident light Iᵣ may be refracted at a surface of the medium TL and then reflected from the road surface RS.

In an ideal case, reflected light Iᵣ obtained by reflection of the incident light Iᵢ from the road surface RS may approximate a Lambertian distribution. An intensity of the reflected light Iᵣ may be relatively uniform over a wide observation angle. The intensity of the reflected light Iᵣ may locally vary significantly depending on roughness, micro-damage, aging, and/or contamination of the road surface. Intrinsic absorption characteristics of the medium TL may be distorted based on variations in the intensity of the reflected light Iᵣ.

Referring to FIG. 2B, in the case of thin-film surface reflection, the incident light Iᵢ may be directly reflected from the surface of the medium TL. The thin-film surface reflection may occur when the surface of the medium TL is smooth.

Referring to FIG. 2C, in the case of road-surface-thin-film-surface reflection, the incident light Iᵢ may be refracted at the surface of the medium TL and then reflected from the road surface RS as first reflected light Iᵣ₁. The incident light Iᵢ may be reflected as second reflected light Iᵣ₂ directly from the surface of the medium TL. The first reflected light Iᵣ₁ and the second reflected light Iᵣ₂ may be reflected simultaneously or sequentially.

A technique for detecting a road surface condition may irradiate a plurality of wavelengths onto the road surface and determine the road surface condition based on an absolute value of reflected light or a relative difference between wavelengths. The detection technique may determine the road surface condition based on the fact that water and ice have different absorption characteristics at a specific wavelength. An absolute intensity of the reflected light may vary depending on material, a degree of wear, a contamination state, and/or surface roughness of the road surface.

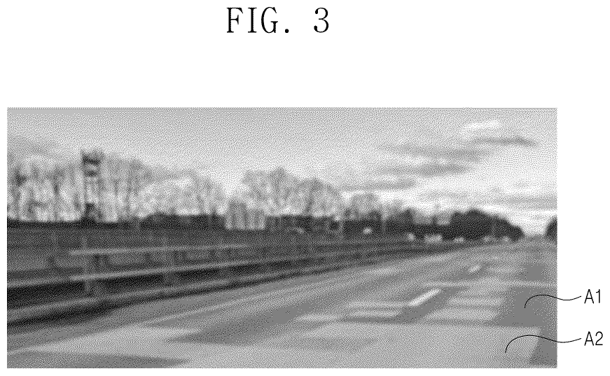

FIG. 3 is an example illustrating a difference in reflectance caused by road repair.

Referring to FIG. 3, it can be confirmed that a reflectance in a dark region A1 is lower than a reflectance in a bright region A2. Diffuse-reflection components of a road may be changed depending on an asphalt type, an age since construction, a degree of aging, micro-damage, and/or a degree of contamination. Even for the same medium, a road may exhibit a non-uniform absolute value of reflected light and a non-uniform difference between wavelengths.

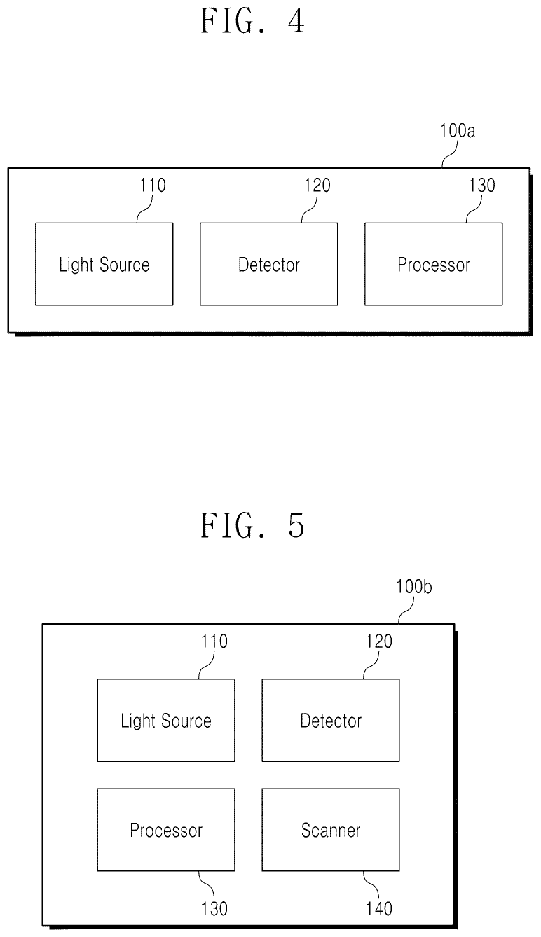

FIG. 4 illustrates a road surface condition detection apparatus according to one embodiment of the present disclosure. Referring to FIG. 4, the road surface condition detection apparatus 100a may include a light source 110, a detector 120, and a processor 130.

In one embodiment, the light source 110 may irradiate light onto the road surface. The light source 110 may irradiate short-wavelength infrared (SWIR) light.

In one embodiment, the light source 110 may include a collimator lens. The light source 110 may convert emitted light into parallel light having a predetermined diameter based on the collimator lens. The light source 110 may minimize divergence of the light by using the parallel light. The light source 110 may concentrate energy of the light by using the parallel light.

In one embodiment, the light source 110 may include a first light source and a second light source. The first light source may output light in a first wavelength band. The first wavelength band may be selected to exhibit inversion of absorption characteristics that are opposite for water and ice. In the first wavelength band, an absorption coefficient of ice may be relatively greater than an absorption coefficient of water. For example, the first wavelength band may include 1550 nm. However, embodiments of the present invention are not limited thereto.

In one embodiment, the second light source may output light in a second wavelength band that is different from the first wavelength band. In the second wavelength band, the absorption coefficient of ice may be relatively smaller than the absorption coefficient of water. For example, the second wavelength band may include 940 nm or 1380 nm. However, embodiments of the present invention are not limited thereto.

In one embodiment, the light source 110 may selectively operate in either a pulse mode or a continuous wave (CW) mode. For example, the light source 110 may selectively operate a mode based on a time period. The light source 110 may operate in the pulse mode during a daytime period in which sunlight interference is strong. The light source 110 may operate in the continuous wave mode during a nighttime period.

In one embodiment, the light source 110 may irradiate a plurality of lights sequentially. The second light source may output the light in the second wavelength band after the first light source outputs the light in the first wavelength band.

In one embodiment, the detector 120 may receive (detect) reflected light. The reflected light may be light irradiated from the light source 110 and reflected by the road surface. The detector 120 may receive (detect) first reflected light obtained by reflection of the light in the first wavelength band. The detector 120 may receive second reflected light obtained by reflection of the light in the second wavelength band.

In one embodiment, the detector 120 may include an image sensor. The image sensor may include a zoom lens or a telephoto lens. The image sensor may observe a fine region of a distant road surface with high resolution by using the zoom lens. The image sensor may intensively scan a forward road surface by using the telephoto lens. Performance of the zoom lens or the telephoto lens of the image sensor may be determined based on a detection distance.

In one embodiment, the image sensor may separate the light in the first wavelength band and the light in the second wavelength band based on a wavelength-selective filter. The image sensor may selectively transmit only wavelengths in the first wavelength band and the second wavelength band based on a multiband-pass filter.

In one embodiment, the image sensor may operate in either a gated mode or a non-gated mode. The gated mode may be a mode that controls an exposure time of the image sensor in correspondence with an emission timing of the light source 110. The non-gated mode may be a mode that acquires ambient-light information by opening a shutter of the image sensor continuously or at a preset period irrespective of pulse operation of the light source 110.

In one embodiment, the processor 130 may include one or more processors. The processor 130 may control the light source 110 and the detector 120. The processor 130 may control an emission timing of the light source 110 and a light-receiving timing of the detector 120. The processor 130 may generate a synchronization signal that synchronizes the emission timing and the light-receiving timing. The processor 130 may transmit the synchronization signal to the light source 110 and the detector 120.

In one embodiment, the processor 130 may control the first light source and the second light source such that output light intensities thereof are maintained constant without varying over time. For example, the processor 130 may adjust a driving current of the light source 110 based on an internal feedback circuit. The processor 130 may control the first light source and the second light source such that output wavelengths thereof are maintained constant without varying over time. For example, the processor 130 may maintain an operating temperature of the light source 110 constant based on a temperature control module. However, embodiments of the present invention are not limited thereto.

In one embodiment, the processor 130 may detect whether the road surface is frozen based on the reflected light. The processor 130 may normalize the first reflected light and the second reflected light with respect to a dry road surface. The processor 130 may calculate an intensity ratio between the normalized first reflected light and the normalized second reflected light. The processor 130 may determine whether the road surface is frozen by comparing the calculated intensity ratio with a reference value.

In one embodiment, the processor 130 may include a machine learning (ML) model. The machine learning model may be trained based on detection data and determination results. For example, the machine learning (ML) model may include at least one of a convolutional neural network (CNN) or a vision transformer (ViT).

In one embodiment, the processor 130 may transmit a control signal to a de-icing (snow-removal) system based on whether the road surface is frozen. The control signal may include information on whether freezing occurs and a freezing location.

FIG. 5 illustrates a road surface condition detection apparatus 100b according to another embodiment of the present disclosure. Referring to FIG. 5, the road surface condition detection apparatus 100b may include a light source 110, a detector 120, a processor 130, and a scanner 140. Descriptions of the light source 110, the detector 120, and the processor 130 which are identical to those of FIG. 4 will be omitted to avoid redundancy.

The scanner 140 may move the light source 110 and the detector 120. The scanner 140 may move the light source 110 and the detector 120 by a predetermined distance under control of the processor 130. The scanner 140 may include at least one of a galvanometer, a rotating mirror, or a MEMS mirror. The scanner 140 may be configured to perform two-axis scanning.

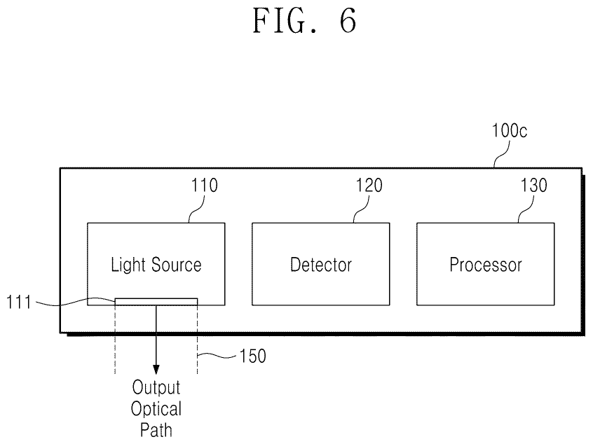

FIG. 6 illustrates a road surface condition detection apparatus 100c according to another embodiment of the present disclosure. Referring to FIG. 6, the road surface condition detection apparatus 100c may include a light source 110, a detector 120, a processor 130, and a Brewster-reflection-based surface determination channel 150. Descriptions of the light source 110, the detector 120, and the processor 130 which are identical to those of FIG. 4 and FIG. 5 will be omitted to avoid redundancy.

The Brewster-reflection-based surface determination channel 150 may determine whether freezing occurs by using surface reflection characteristics of a medium formed on the road surface.

The Brewster-reflection-based surface determination channel 150 may be configured such that the light source 110 irradiates linearly polarized light toward the road surface. The light source 110 may include a polarizer 111. The polarizer 111 may be disposed on an output optical path. The polarizer 111 may output the linearly polarized light in a P-polarization state. The P-polarization state may have an electric-field component parallel to a plane of incidence.

The Brewster-reflection-based surface determination channel 150 may be configured such that at least one of the light source 110 or the detector 120 includes at least one of an angle adjuster or a height adjuster. The angle adjuster may adjust an irradiation angle. The height adjuster may adjust a relative installation height with respect to the road surface. At least one of the angle adjuster or the height adjuster may cause the linearly polarized light to be incident at an incidence angle. The incidence angle may correspond to a Brewster angle of the medium formed on the road surface.

The detector 120 may detect a reflected component of the P-polarized light. The P-polarized light may be reflected from the road surface under a Brewster reflection condition.

The processor 130 may determine whether the medium formed on the road surface is water or ice. The processor 130 may determine the medium based on whether an intensity of the reflected component of the P-polarized light is extinguished or reduced.

The processor 130 may combine a determination result from the Brewster-reflection-based surface determination channel 150 with a freezing determination result based on absorption-characteristic inversion and normalization. The processor 130 may determine whether the road surface is frozen in an auxiliary manner or in a cross-validation manner based on the determination result from the Brewster-reflection-based surface determination channel 150.

The embodiment of FIG. 6 may be combined with the embodiment of FIG. 5.

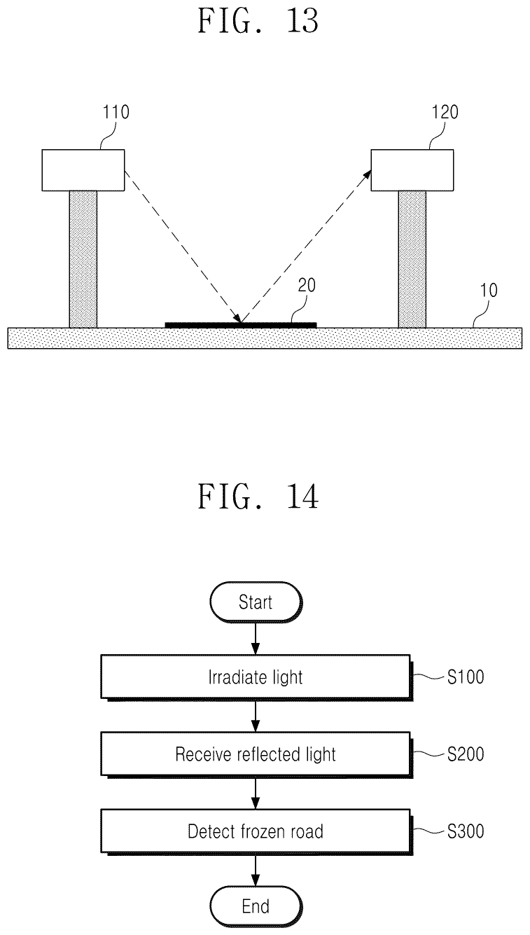

The structures of FIG. 4, FIG. 5, and FIG. 6 may be used as an integrated type in which the light source 110 and the detector 120 are integrated based on installation environments and conditions. The structures of FIG. 4, FIG. 5, and FIG. 6 may be used as a separate type in which the light source 110 and the detector 120 are separated from each other based on the installation environments and conditions. An example in which the light source 110 and the detector 120 are separated from each other is illustrated in FIG. 13.

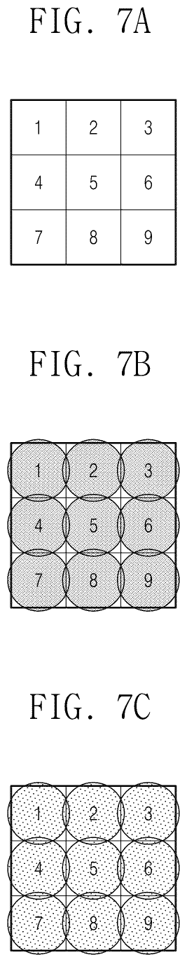

FIG. 7A illustrates an example of dividing a road surface according to one embodiment of the present disclosure. FIG. 7B illustrates an example of irradiating light in a first wavelength band onto the road surface of FIG. 7A. FIG. 7C illustrates an example of irradiating light in a second wavelength band onto the road surface of FIG. 7A. FIGS. 7A to 7C will be described together with FIGS. 5 and 6.

FIGS. 7A to 7C may represent detection signals obtained for a dry road surface. Measurement results shown in FIGS. 7A to 7C may be used as calibration data for removing deviations caused by a road-surface material or a thin film. The processor 130 may normalize a real-time detection signal based on the measurement results in FIGS. 7A to 7C.

Referring to FIG. 7A, the processor 130 may divide the road surface into a plurality of unit cells. The road surface may refer to the road surface in an image acquired by the detector 120. For example, the processor 130 may divide the road surface into nine unit cells in a 3×3 arrangement. However, embodiments of the present invention are not limited thereto, and the number of the unit cells may be changed.

In one embodiment, the processor 130 may set a size or an area of each unit cell to correspond to a diameter of the parallel light irradiated from the light source 110 or an irradiated area by the parallel light. The size or the area of each unit cell may include or correspond to a region irradiated by the parallel light.

In one embodiment, the processor 130 may control the light source 110 and the detector 120 to sequentially scan each unit cell. The processor 130 may control the scanner 140 to two-dimensionally scan, for the plurality of unit cells, an irradiation point of the light source 110 and the detector 120. The processor 130 may determine whether each unit cell is frozen.

In one embodiment, the processor 130 may control the detector 120, for each unit cell, to receive the reflected light for a period corresponding to at least one of an integration time or an exposure time and then move to a next unit cell. The integration time or the exposure time may refer to a time interval during which the detector 120 generates a signal based on accumulation of incident light. For example, the processor 130 may control, after the detector 120 receives the reflected light for a predetermined period for a first cell of FIG. 7A, the light source 110 and the detector 120 to move to a second cell of FIG. 7A.

In one embodiment, the processor 130 may control the first reflected light and the second reflected light to be sequentially acquired for each unit cell. For example, the light source 110 may irradiate, onto the first cell of FIG. 7A, the light in the first wavelength band (FIG. 7B) and the light in the second wavelength band (FIG. 7C) in a time-division manner. The detector 120 may sequentially receive the first reflected light of FIG. 7B and the second reflected light of FIG. 7C. Based on receiving reflected light of a plurality of wavelengths for the first cell, the processor 130 may control the light source 110 and the detector 120 to move to the second cell of FIG. 7A.

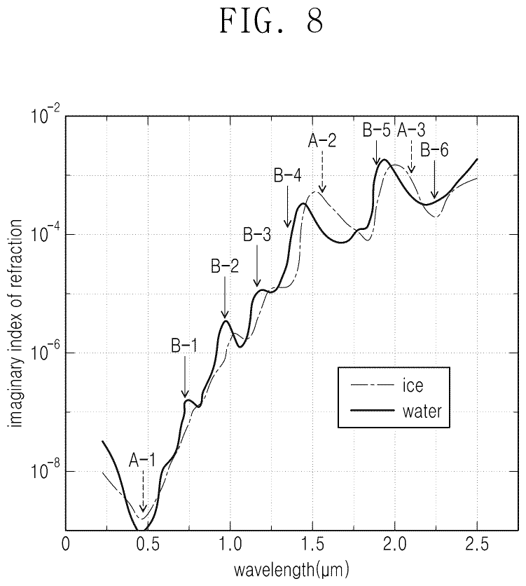

FIG. 8 is a graph illustrating changes in an imaginary part of a complex refractive index of water and ice according to wavelength. FIG. 8 may represent results obtained by the road surface condition detection apparatus 100a of FIG. 4. Referring to FIG. 8, a horizontal axis may represent a wavelength, and a vertical axis may represent the imaginary part of the complex refractive index. The imaginary part of the complex refractive index may be proportional to an optical absorption coefficient of a material. A larger value of the imaginary part of the complex refractive index may indicate that more optical energy at the corresponding wavelength is absorbed. Points A-1 to A-3 and B-1 to B-6 may indicate maxima or intersection points of absorption characteristics of water and ice.

Referring to FIG. 8, it can be confirmed that water and ice have different absorption characteristics. Water and ice may exhibit inversion of absorption characteristics in which a relative magnitude relationship of absorption reverses around a specific wavelength point.

For example, point A-2 of FIG. 8 may represent spectroscopic absorption characteristics of water and ice in the first wavelength band, such as 1550 nm. At point A-2, it can be observed that an imaginary-part value of a complex refractive index of ice is higher than that of water. In the wavelength band at point A-2, ice may absorb optical energy more strongly than water. The detector 120 may measure that, in the wavelength band at point A-2, a frozen road surface has reflected energy of a relatively lower intensity than a wet road surface.

For example, point B-2 of FIG. 8 may represent spectroscopic absorption characteristics of water and ice in the second wavelength band, such as 940 nm. At point B-2, it can be observed that an imaginary-part value of a complex refractive index of ice is lower than that of water. In the wavelength band at point B-2, ice may absorb optical energy more weakly than water. The detector 120 may measure that, in the wavelength band at point B-2, the wet road surface has reflected energy of a relatively lower intensity than the frozen road surface.

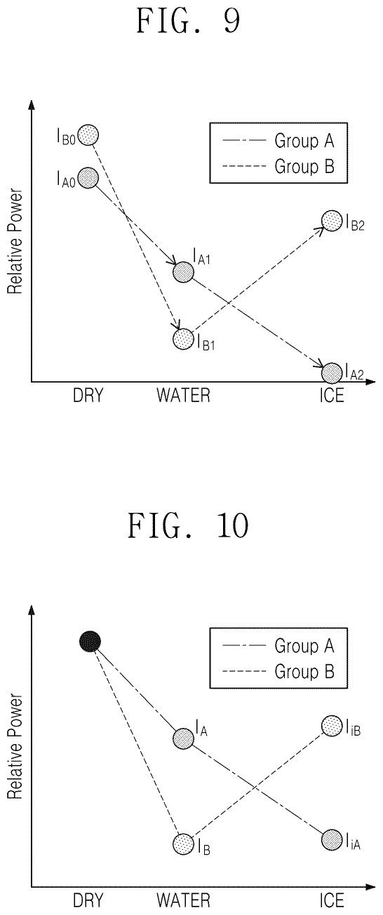

FIG. 9 is a graph illustrating results measured for a dry road surface by the road surface condition detection apparatus according to one embodiment of the present disclosure. FIG. 9 may represent results obtained by the road surface condition detection apparatus 100a of FIG. 4. Referring to FIG. 9, an x-axis may represent a state, and a y-axis may represent an intensity of reflected light. FIG. 9 will be described together with FIG. 4.

In one embodiment, I_{A0} and I_{B0} of FIG. 9 may represent an intensity of first reflected light corresponding to the first wavelength band and an intensity of second reflected light corresponding to the second wavelength band for a dry road surface, respectively. I_{A0} and I_{B0} may differ from each other depending on a road surface condition. In the case of FIG. 9, it can be confirmed that I_{B0} has a value larger than that of I_{A0}. However, embodiments of the present invention are not limited thereto, and I_{A0} may have a value larger than that of I_{B0}.

In one embodiment, I_{A1} and I_{B1} of FIG. 9 may represent an intensity of first reflected light and an intensity of second reflected light for a road surface on which water is present. The processor 130 may determine a road surface condition based on a comparison between a value of I_{A1} and a value of I_{B1}.

In one embodiment, I_{A2} and I_{B2} of FIG. 9 may represent an intensity of first reflected light and an intensity of second reflected light for a road surface on which ice is present. The processor 130 may determine a road surface condition based on a comparison between a value of I_{A2} and a value of I_{B2}.

FIG. 10 is a graph illustrating normalized values of measurement results for the dry road surface of FIG. 9. FIG. 10 may represent results obtained by the road surface condition detection apparatus 100a of FIG. 4. Referring to FIG. 10, an x-axis may represent a state, and a y-axis may represent an intensity of reflected light. FIG. 10 will be described together with FIG. 4.

In one embodiment, the processor 130 may perform data normalization on I_{A0} and I_{B0} for the dry road surface. The processor 130 may map I_{A0} and I_{B0} to a same reference value based on appropriate weights applied to I_{A0} and I_{B0}. For example, the reference value may be 1. However, embodiments of the present invention are not limited thereto. The processor 130 may remove differences in absolute reflectance caused by a road surface material, roughness, and contamination based on the normalization. The processor 130 may extract only intrinsic absorption components of a material due to a medium layer based on the normalization.

In one embodiment, the processor 130 may determine a road surface condition based on an intensity of normalized first reflected light corresponding to the first wavelength band and an intensity of normalized second reflected light corresponding to the second wavelength band. The processor 130 may calculate a ratio between the normalized first reflected light and the normalized second reflected light based on Equation 1 below. However, embodiments of the present invention are not limited thereto, and the processor 130 may calculate the ratio as a reciprocal of Equation 1 below.

$$R = \frac{{I}_{B}}{{I}_{A}} \qquad [ Equation 1 ]$$

In Equation 1, R represents a ratio between the normalized first reflected light and the normalized second reflected light, I_{A} represents an intensity of the normalized first reflected light, and I_{B} represents an intensity of the normalized second reflected light.

In one embodiment, the processor 130 may determine a road surface condition by comparing the ratio between the normalized first reflected light and the normalized second reflected light with a reference value. The processor 130 may determine the road surface to be a dry road surface when the ratio between the normalized first reflected light and the normalized second reflected light is equal to the reference value (for example, R=1). The processor 130 may determine the road surface to be an ice-covered road surface, that is, black ice, when the ratio is greater than the reference value (for example, R>1). The processor 130 may determine the road surface to be a wet road surface when the ratio is smaller than the reference value (for example, R<1).

Referring to FIG. 10, in a case of water (a water film), it can be confirmed that an intensity of the normalized first reflected light is greater than an intensity of the normalized second reflected light. In this case, the processor 130 may determine the corresponding road surface to be a wet road surface having a water film based on the ratio between the normalized first reflected light and the normalized second reflected light being smaller than the reference value of 1.

Referring to FIG. 10, in a case of ice (an ice film), it can be confirmed that an intensity of the normalized first reflected light is smaller than an intensity of the normalized second reflected light. In this case, the processor 130 may determine the corresponding road surface to be an ice-covered road surface having an ice film, that is, black ice, based on the ratio being greater than the reference value of 1.

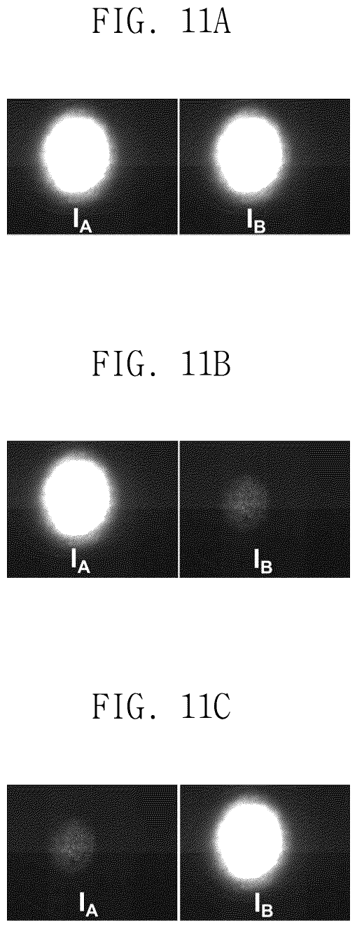

In one embodiment, the processor 130 may perform independent analysis for each unit cell obtained by dividing an observation region into a plurality of grids. The processor 130 may generate data such as that shown in FIGS. 10 and 11 for each cell. The processor 130 may sequentially perform a normalization process for each cell. The processor 130 may determine a road surface condition for each cell. The processor 130 may map a distribution of a surface layer film on the road surface for a plurality of cells within a target region based on determination results of the road surface condition.

In one embodiment, the road surface condition detection apparatus 100a may periodically perform measurement and normalization under dry weather conditions without rainfall or snowfall. The road surface condition detection apparatus 100a may update changes in reflectance of the road surface itself in real time based on the periodic measurement and normalization.

In one embodiment, the processor 130 may calculate a thickness, a concentration, and/or absorption characteristics of a thin film based on changes in an intensity of the first reflected light and changes in an intensity of the second reflected light derived from a measured value of the ratio R. The processor 130 may generate a model that inversely estimates the thickness, the concentration, and/or the absorption characteristics of the thin film.

FIG. 11A is a measurement image of first reflected light and second reflected light for the normalized dry road surface of FIG. 10. FIG. 11B is a measurement image of first reflected light and second reflected light for a normalized water film of FIG. 10. FIG. 11C is a measurement image of first reflected light and second reflected light for a normalized ice film of FIG. 10.

Referring to FIG. 11A, an image of the first reflected light and an image of the second reflected light may be measured to have a same level of brightness or detected energy. The processor 130 may perform normalization such that the first reflected light in the first wavelength band I_{A} and the second reflected light in the second wavelength band I_{B} obtained from the dry road surface have a same value.

Referring to FIG. 11B, in a case of a road surface on which a water film is formed, an image of the first reflected light may appear brighter than an image of the second reflected light. An absorption coefficient of water in the water film may be greater in the second wavelength band I_{B} than in the first wavelength band I_{A}. Based on the fact that the second reflected light is absorbed by water more than the first reflected light, the image of the second reflected light may be formed darker than the image of the first reflected light.

Referring to FIG. 11C, in a case of a road surface on which an ice film is formed, an image of the second reflected light may appear brighter than an image of the first reflected light. An absorption coefficient of ice in the ice film may be greater in the first wavelength band I_{A} than in the second wavelength band I_{B}. Based on the fact that the first reflected light is absorbed by ice more than the second reflected light, the image of the first reflected light may be formed darker than the image of the second reflected light.

In one embodiment, the processor 130 may determine a road surface condition based on a relative brightness pattern of the first reflected light and the second reflected light. The processor 130 may distinguish among a dry road surface, a water-film road surface, and an ice-film road surface based on comparing a brightness (intensity) ratio or a difference value of the reflected light with a preset threshold value.

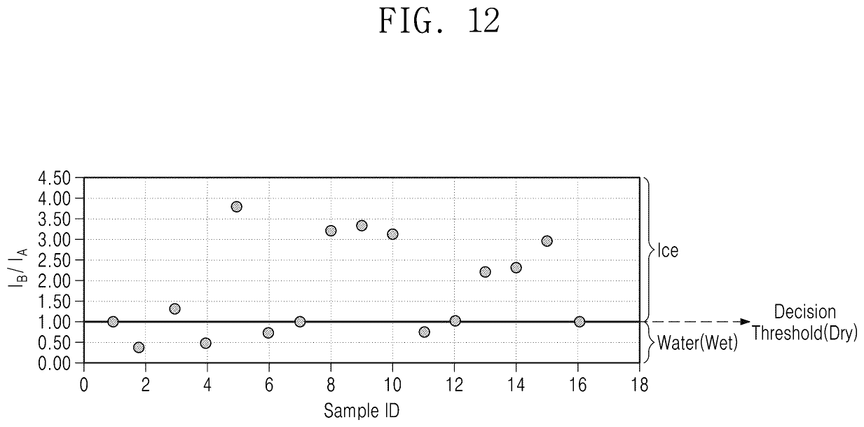

FIG. 12 is a graph illustrating an example of determining a road surface condition based on an intensity ratio of reflected light according to one embodiment of the present disclosure. Referring to FIG. 12, a horizontal axis may represent a sample identifier, and a y-axis may represent a ratio of intensities of reflected light. In FIG. 12, the intensity ratio of the reflected light may use an intensity of the second reflected light relative to an intensity of the first reflected light. FIG. 12 will be described together with FIGS. 4, 5, and/or 6.

In one embodiment, the processor 130 may classify a road surface condition based on a decision threshold. The processor 130 may determine the corresponding road surface to be a wet road surface having a water film based on an intensity ratio smaller than the decision threshold (in FIG. 12, samples 2, 4, 6, and 11). The processor 130 may determine the corresponding road surface to be a dry road surface based on an intensity ratio equal to the decision threshold (in FIG. 12, samples 1, 7, 12, and 16). The processor 130 may determine the corresponding road surface to be an ice-covered road surface having an ice film, that is, black ice, based on an intensity ratio greater than the decision threshold (in FIG. 12, samples 3, 5, 8, 9, 10, 13, 14, and 15).

FIG. 13 is a schematic diagram illustrating an application example of a road surface condition detection apparatus according to one embodiment of the present disclosure. Referring to FIG. 13, the road surface condition detection apparatus may include a light source 110 and a detector 120.

In one embodiment, the light source 110 may irradiate light to a target region 20 of a road 10. The detector 120 may receive reflected light reflected by the target region 20 from the light irradiated by the light source 110. The road surface condition detection apparatus may detect whether the road 10 is frozen based on the reflected light.

In one embodiment, the light source 110 and the detector 120 may be installed on a pole and configured to face downward toward the road 10. The light source 110 and the detector 120 may be installed adjacent to each other side by side. The light source 110 and the detector 120 may be disposed to face each other with the target region 20 therebetween. In FIG. 13, an example is shown in which the light source 110 and the detector 120 are disposed to face each other. However, embodiments of the present disclosure are not limited thereto. In another embodiment, the road surface condition detection apparatus may be mounted on a vehicle for an advanced driver assistance system (ADAS) and autonomous driving. In yet another embodiment, the road surface condition detection apparatus may be installed to monitor a runway or a bridge deck.

FIG. 14 is a flowchart illustrating a method for operating a road surface condition detection apparatus according to one embodiment of the present disclosure. FIG. 14 will be described together with FIGS. 4, 5, and/or 6.

Referring to FIGS. 4, 5, 6, and/or 14, in step S100, the light source 110 may irradiate light. In step S200, the detector 120 may receive reflected light. In step S300, the processor 130 may detect whether the road is frozen.

In one embodiment, the light source 110 may irradiate light to a road surface. The light source 110 may irradiate short-wavelength infrared light. The light source 110 may convert the irradiated light into parallel light having a predetermined diameter based on a collimator lens.

In one embodiment, the light source 110 may include a first light source and a second light source. The first light source may output light in a first wavelength band selected to exhibit inversion of absorption characteristics between water and ice. In the first wavelength band, an absorption coefficient of ice may be relatively greater than an absorption coefficient of water. For example, the first wavelength band may include 1550 nm. However, embodiments of the present disclosure are not limited thereto.

In one embodiment, the second light source may output light in a second wavelength band different from the first wavelength band. In the second wavelength band, an absorption coefficient of ice may be relatively smaller than an absorption coefficient of water. For example, the second wavelength band may include 940 nm or 1380 nm. However, embodiments of the present disclosure are not limited thereto.

In one embodiment, the light source 110 may sequentially irradiate a plurality of lights. The second light source may output the light in the second wavelength band after the first light source outputs the light in the first wavelength band.

In one embodiment, the detector 120 may receive reflected light reflected by the road surface from the light irradiated by the light source 110. The detector 120 may receive first reflected light reflected from the light in the first wavelength band and second reflected light reflected from the light in the second wavelength band.

In one embodiment, the processor 130 may detect whether the road is frozen based on the reflected light. The processor 130 may normalize the first reflected light and the second reflected light with respect to a dry road surface. The processor 130 may calculate an intensity ratio between the normalized first reflected light and the normalized second reflected light. The processor 130 may determine whether the road surface is frozen by comparing the calculated intensity ratio with a reference value.

In one embodiment, the processor 130 may combine a determination result from a Brewster-reflection-based surface determination channel 150 with a freezing determination result based on absorption inversion and normalization. The processor 130 may determine whether the road surface is frozen in an auxiliary manner or a cross-validation manner based on the determination result from the Brewster-reflection-based surface determination channel 150.

According to one or more embodiments of the present disclosure, reflectance variation caused by external factors including a road material, surface roughness, aging, and contamination may be removed by normalizing reflected light for each wavelength based on a reference value of a dry surface. Accordingly, the present disclosure may improve reliability of black ice detection.

In addition, one or more embodiments of the present disclosure may provide an absolute determination criterion for water and ice that are difficult to distinguish. Accordingly, one or more embodiments of the present disclosure may stably distinguish water and ice regardless of external variables such as an illuminance change, a distance change, and a road paving method change, based on the absolute determination criterion.

In addition, since one or more embodiments of the present disclosure may periodically update a normalization criterion, the one or more embodiments may automatically adapt to reflectance changes caused by road damage, asphalt construction, and surface repair even during long-term use.

Furthermore, since normalization and ratio calculation in one or more embodiments of the present disclosure are implemented with simple arithmetic operations, high-speed computation may be possible and real-time measurement and warning may be possible while a vehicle is traveling. Accordingly, the present disclosure may be integrated into a road friction prediction system of an autonomous vehicle or an ADAS.

Moreover, the present disclosure may be implemented in various forms such as a vehicle-mounted type, a roadside fixed type, and a drone-based remote measurement system. The present disclosure may also be applied to black ice monitoring over a wide area such as an airport runway, a highway, and a bridge deck. The present disclosure may maintain accuracy even when an installation position changes or a distance change occurs.

Although embodiments of the present disclosure have been described in detail above, the scope of the present disclosure is not limited thereto, and various modifications and improvements by those skilled in the art using the basic concept of the present disclosure also fall within the scope of the present disclosure.

1. A road surface condition detection apparatus, comprising:
a light source configured to irradiate light onto a road surface;
a detector configured to receive reflected light reflected by the road surface in response to the light irradiated from the light source; and
a processor configured to control the light source and the detector and to detect whether the road surface is frozen based on the reflected light,
wherein the light source comprises:
a first light source configured to output light in a first wavelength band in which an absorption coefficient of ice is greater than an absorption coefficient of water, the first wavelength band being selected to exhibit inversion of absorption characteristics between water and ice; and
a second light source configured to output light in a second wavelength band different from the first wavelength band, and
wherein the processor is configured to:
normalize first reflected light corresponding to the first wavelength band and second reflected light corresponding to the second wavelength band based on a reference reflectance value obtained from a dry road surface;
calculate an intensity ratio between the normalized first reflected light and the normalized second reflected light; and
determine whether the road surface is frozen by comparing the calculated intensity ratio with a reference value, and
wherein the processor normalizes the first reflected light and the second reflected light by applying weights such that the first reflected light and the second reflected light obtained from the dry road surface are mapped to a same value.

2. The road surface condition detection apparatus of claim 1,
wherein the light source irradiates short-wavelength infrared (SWIR) light, and
wherein the short-wavelength infrared light is parallel light having a predetermined diameter.

3. The road surface condition detection apparatus of claim 1,
wherein the first wavelength band includes 1550 nm, and
wherein the second wavelength band includes 940 nm or 1380 nm.

4. The road surface condition detection apparatus of claim 1,
wherein the reflected light includes first reflected light corresponding to light in the first wavelength band and second reflected light corresponding to light in the second wavelength band.

5. The road surface condition detection apparatus of claim 1,
wherein the processor controls output intensities of the first light source and the second light source to be maintained constant over time.

6. The road surface condition detection apparatus of claim 1,
wherein the processor controls output wavelengths of the first light source and the second light source to be maintained constant over time.

7. The road surface condition detection apparatus of claim 1,
further comprising a scanner configured to move the light source and the detector,
wherein the processor is configured to:
divide the road surface into a plurality of unit cells; and
control the scanner to two-dimensionally scan irradiation points of the light source and the detector for the plurality of unit cells while determining whether each unit cell is frozen.

8. The road surface condition detection apparatus of claim 7,
wherein the processor sets a size or an area of each unit cell to correspond to a diameter of the parallel light irradiated from the light source or an irradiation area by the parallel light.

9. The road surface condition detection apparatus of claim 7,
wherein the processor controls the light source and the detector to sequentially scan the unit cells, and
controls the detector to receive the reflected light for at least a period corresponding to an integration time or an exposure time for each unit cell before moving to a next unit cell.

10. The road surface condition detection apparatus of claim 1,
wherein the light source operates in a pulse mode or a continuous-wave mode, and
wherein the second light source outputs light in the second wavelength band after the first light source outputs light in the first wavelength band.

11. The road surface condition detection apparatus of claim 1,
wherein the detector comprises an image sensor, and
wherein the image sensor separates light in the first wavelength band and light in the second wavelength band based on a wavelength-selective filter.

12. The road surface condition detection apparatus of claim 11,
wherein the image sensor comprises a zoom lens or a telephoto lens.

13. The road surface condition detection apparatus of claim 11,
wherein the processor controls the image sensor to selectively operate in one of a gated mode and a non-gated mode, and
wherein the gated mode controls an exposure time of the image sensor in correspondence with an emission timing of the light source.

14. A road surface condition detection apparatus, comprising:
a light source configured to irradiate light onto a road surface;
a detector configured to receive reflected light reflected by the road surface in response to the light irradiated from the light source; and
a processor configured to control the light source and the detector and to detect whether the road surface is frozen based on the reflected light,
wherein the light source comprises:
a first light source configured to output light in a first wavelength band in which an absorption coefficient of ice is greater than an absorption coefficient of water, the first wavelength band being selected to exhibit inversion of absorption characteristics between water and ice; and
a second light source configured to output light in a second wavelength band different from the first wavelength band, and
wherein the processor is configured to:
normalize first reflected light corresponding to the first wavelength band and second reflected light corresponding to the second wavelength band based on a reference reflectance value obtained from a dry road surface;
calculate an intensity ratio between the normalized first reflected light and the normalized second reflected light; and
determine whether the road surface is frozen by comparing the calculated intensity ratio with a reference value,
wherein the road surface condition detection apparatus further comprises a Brewster-reflection-based surface determination channel configured to determine whether the road surface is frozen using surface reflection characteristics of a medium formed on the road surface.

15. The road surface condition detection apparatus of claim 14,
wherein the Brewster-reflection-based surface determination channel is configured such that the light source irradiates linearly polarized light toward the road surface, and
wherein the light source includes a polarizer disposed on an output optical path.

16. The road surface condition detection apparatus of claim 15,
wherein the linearly polarized light is P-polarized light having an electric-field component parallel to an incident plane, and is incident at an incident angle corresponding to a Brewster angle of the medium formed on the road surface.

17. The road surface condition detection apparatus of claim 16,
wherein, to form the incident angle corresponding to the Brewster angle, at least one of the light source or the detector comprises at least one of:
an angle adjuster configured to adjust an irradiation angle; or
a height adjuster configured to adjust a relative installation height with respect to the road surface.

18. The road surface condition detection apparatus of claim 16,
wherein the detector detects a P-polarized reflection component reflected from the road surface under a Brewster-reflection condition, and
wherein the processor determines whether the medium formed on the road surface is water or ice based on whether an intensity of the P-polarized reflection component vanishes or decreases.

19. The road surface condition detection apparatus of claim 14,
wherein the processor combines a determination result from the Brewster-reflection-based surface determination channel with a freezing determination result based on absorption inversion and normalization to determine whether the road surface is frozen in an auxiliary or cross-validation manner.

20. The road surface condition detection apparatus of claim 7,
wherein the processor:
determines whether each of the plurality of unit cells is frozen; and
maps a distribution of a surface layer film on the road surface based on determination results for the plurality of unit cells.

21. A road surface condition detection apparatus, comprising:
a light source configured to irradiate light onto a road surface;
a detector configured to receive reflected light reflected by the road surface in response to the light irradiated from the light source; and
a processor configured to control the light source and the detector and to detect whether the road surface is frozen based on the reflected light,
wherein the light source comprises:
a first light source configured to output light in a first wavelength band in which an absorption coefficient of ice is greater than an absorption coefficient of water, the first wavelength band being selected to exhibit inversion of absorption characteristics between water and ice; and
a second light source configured to output light in a second wavelength band different from the first wavelength band, and
wherein the processor is configured to:
normalize first reflected light corresponding to the first wavelength band and second reflected light corresponding to the second wavelength band based on a reference reflectance value obtained from a dry road surface;
calculate an intensity ratio between the normalized first reflected light and the normalized second reflected light; and
determine whether the road surface is frozen by comparing the calculated intensity ratio with a reference value, and
wherein the processor periodically performs measurement and normalization under dry weather conditions and updates the reference reflectance value in real time based on periodic measurement.
